(19) United States
(12) Patent Application Publication
(10) Pub. No.: US 2026/0114592 A1
(43) Pub. Date: Apr. 30, 2026

(54) COSMETIC APPLICATOR

(71) Applicant: GEKA GmbH, Bechhofen (DE)

(72) Inventor: Margherita di MAURO, Bellusco (IT)

(21) Appl. No.: 19/371,277

(22) Filed: Oct. 28, 2025

(30) Foreign Application Priority Data
Oct. 31, 2024 (DE) 202024106257.4

(51) Int. Cl. A45D 40/26; A61M 35/00

(52) U.S. Cl. CPC A45D 40/26; A61M 35/003

(57) ABSTRACT

An applicator for applying a cosmetic or pharmaceutical product includes a stalk and an applicator. The applicator element is attached to the stalk. The applicator element has, on a front side facing away from the stalk, an applicator surface which, when projected onto a plane, has a cam-shaped, drop-shaped, egg-shaped, or pear-shaped contour line extending on all sides beyond an imaginary extension of the stalk and is continuously convexly curved, concavely curved, or flat in a direction along an imaginary line between first and second extreme points.

### PRIORITY

This application is a U.S. Non-Provisional application which claims priority to German Patent Application No. 202024106257.4 which was filed on Oct. 31, 2024, the contents of which are hereby incorporated by reference.

### TECHNICAL INFORMATION

The disclosure relates to a cosmetic applicator for applying cosmetic material.

### BACKGROUND

In the application of medical or cosmetic products such as creams, ointments, or makeup, cosmetic applicators are often used. Conventional cosmetic applicators generally comprise a handle or stalk as well as a section used to apply the product to be applied from the desired area through stroking, dabbing, or rotating motions. This section of a cosmetic applicator is also referred to as the applicator element. Depending on the type of product to be applied, different designs can be considered regarding the geometry and material of the cosmetic applicator or the applicator element.

### SUMMARY

For the application of lip gloss and other cosmetic, hygiene, and medical products that are typically applied to the skin, brush-like cosmetic applicators are often used. These include a stalk and a number of bristles at the free end of the applicator. The bristles serve as the applicator element.

To apply the product from the desired area, the bristles must first be wetted with the product to be applied. For this purpose, they are usually dipped into a container holding the product. However, the amount that adheres to the bristles can only be dosed to a limited extent. While it is possible to dip the applicator into the container so that only the tips of the applicator element come into contact with the product, depending on the viscosity of the product, it has been determined that unwanted coating of the entire applicator element can occur. With highly viscous products, the individual bristles and/or the spaces between them can, due to capillary effects, become coated with the product along their entire length, even if the bristles are not completely submerged in the product within the container.

For this reason, the bristles are typically wiped off before applying the product, either on a cloth (less commonly) or on a special wiper located on the inside of the container, so that the excess product adhering to the bristles is removed. However, the amount of product remaining on the bristles after wiping is also difficult to dose. As a result, an undesirably large amount of product can be applied. In addition, this can lead to unwanted contamination of the outer surface of the container. While contamination of the container's exterior can be avoided by wiping the applicator on a cloth or similar item, this results in increased consumption of the product and higher waste generation.

Alternatively, the application process can be unnecessarily prolonged if too little or too much product remains on the bristles, requiring the process to be repeated or aborted and/or restarted.

In view of the above, the objective of the disclosure is to provide a cosmetic applicator in which the amount of product to be applied-both the amount absorbed by and released from the applicator element—can be more easily dosed.

According to the disclosure, this problem is solved by the features of the cosmetic applicator disclosed herein.

For this purpose, an applicator for applying a cosmetic or pharmaceutical product is proposed, wherein the applicator comprises a stalk and an applicator element attached thereto. The applicator is characterized in that the applicator element has, on its front side facing away from the stalk, a preferably continuous applicator surface which—when projected onto a plane—has a cam-shaped, drop-shaped, egg-shaped, or pear-shaped contour line. This the contour line extends on all sides beyond the imaginary extension of the stalk and is continuously-preferably over its entire width-convexly curved, concavely curved, or flat along an imaginary line between two extreme points.

“In a plane projection” within the meaning of the disclosure refers to the imaginary projection of the applicator surface onto a flat plane, in cases where the applicator surface itself is not flat. This can be illustrated, for example, by the shadow cast by the applicator surface onto a plane perpendicular to the longitudinal axis of the stalk.

In this way, an applicator can be easily produced that allows for simple and favorable application of the material to be applied, as well as favorable wetting and massaging behavior, both resulting from the simple shape of the applicator surface. It should be noted that the applicator surface is not the only part of the applicator element intended for the designated application. In many cases, it is even preferable for the application and, in particular, the finer “fine-tuning” to be possible with the entire applicator element. This represents another advantage of the applicator according to the disclosure, as it can adapt to the desired applicator behavior on different surfaces and is therefore particularly well suited for use on various roundings on the face.

One embodiment of the applicator provides that the contour line of the applicator surface has its smallest curvature at its first extreme point or in the vicinity of the first extreme point, and its second smallest curvature at its second extreme point or in the vicinity of the second extreme point. The mentioned “vicinity” refers to a circular area with a diameter of no more than 30 mm, and preferably no more than 10 mm, around the extreme point. This generally results in a preferred shape of the applicator surface for improved applicator of the medium to be applied.

Furthermore, it is particularly preferred that the curvature of the contour line at the second extreme point is at least three times, preferably at least five times, greater than the curvature of the contour line at the first extreme point. This also results in a preferred shape of the applicator surface for improved applicator of the medium to be applied.

It is also particularly preferred that the extreme points lie diametrically opposite each other with respect to the extended imaginary central longitudinal axis of the stalk. Thus, it is preferred that the two extreme points are located in exactly opposite directions. Thereby, the applicator element can be designed to be mirror-symmetrical; however, in many cases, it is preferred that the applicator element is not mirror-symmetrical, so that the two extreme points are arranged offset from one another with respect to a central plane.

It is further particularly preferred that the applicator surface, in the direction perpendicular to the imaginary line between the two extreme points, is less curved, not curved, or at least substantially not curved. Thereby the direction perpendicular to the imaginary line refers to the transverse direction of the applicator element. This is indicated by stripes on the applicator surface in the later figures.

It is also particularly preferred that the applicator consists entirely or partially of a TPE (thermoplastic elastomer), or a TPO (thermoplastic polyolefin), or a TPE- or TPO-containing mixture. Due to the preferably rubber-like elasticity achieved by proper formulation, this contributes to the desired application behavior.

Moreover, it is particularly preferred that the applicator surface or the entire applicator element carries at least partially, or ideally entirely, a flocking and/or micro-bristles and/or another type of surface modification. This can positively influence both the wetting behavior and the applicator performance of the applicator. “Micro-bristles” in this context refer to bristles with a length of less than 2 mm, preferably less than 1 mm, and a diameter of less than 5 hundredth of a mm, preferably less than 2 hundredth of a mm.

It is also particularly preferred that the tip of the applicator surface in the area of the first extreme point is shaped and made of a material such that, under the forces typically occurring during application, it can move—e.g., pivot—by at least 2 mm relative to the rest of the applicator element. This again positively influences the application behavior.

Another preferred embodiment of the applicator provides that the applicator element is designed in the form of a shoe. In this case, the applicator element preferably widens uniformly upward from the stalk, forming the ankle area of the shoe. Thereafter, the applicator element widens more strongly on one side (heel area) than on the side diametrically opposite with respect to the extended longitudinal axis L (tongue/flap area). The side that widens more strongly ends lower-toward the stalk—than the diametrically opposite side. The lower-ending side terminates, among other things, at the previously mentioned first extreme point, while the diametrically opposite side preferably terminates, among other things, at a second extreme point. Between the two sides—and thus also between the two extreme points—the applicator surface extends, which essentially functions as the sole of the shoe.

Further functionalities, advantages, and design possibilities become apparent from the figure-based description of the exemplary embodiments.

### BRIEF DESCRIPTION OF THE DRAWINGS

FIG. 1 shows a first exemplary embodiment of the applicator according to the disclosure (with a convexly curved applicator surface) in a three-dimensional view.

FIG. 2 shows a second exemplary embodiment of the applicator according to the disclosure (with a flat applicator surface) in a three-dimensional view.

FIG. 3 shows a first exemplary embodiment of the applicator according to the disclosure (with a concavely curved applicator surface) in a three-dimensional view.

FIG. 4 shows an additional three-dimensional view applicable to all exemplary embodiments of the applicator according to the disclosure.

FIG. 5 shows another three-dimensional view applicable to all exemplary embodiments of the applicator according to the disclosure.

FIG. 6 shows a fourth exemplary embodiment of the applicator according to the disclosure in a top view.

FIG. 7 shows a bottom view applicable to all exemplary embodiments of the applicator according to the disclosure.

### DETAILED DESCRIPTION

### First Embodiment

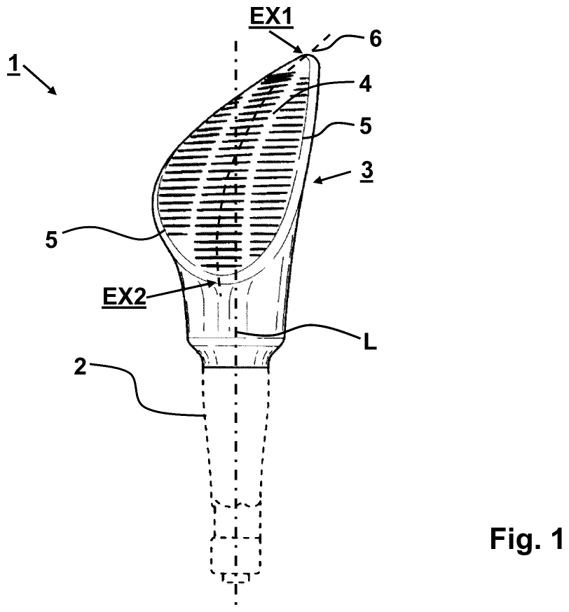

Firstly FIG. 1 shows a preferred exemplary embodiment of the applicator 1 according to the disclosure. The applicator 1 comprises a stalk 2, which is indicated only by dashed lines. This stalk 2 can serve as an additional part for attaching a handle or can itself be the handle that the user holds in the hand or between the fingers to use the applicator 1 as intended. The central longitudinal axis L of the applicator 1 is the central longitudinal axis of the stalk 2, extended imaginatively.

Projecting from the stalk 2, the applicator 1 further comprises an applicator element 3, which carries the applicator surface 4.

On its front side facing away from the stalk 2, the applicator 1 namely has an applicator surface 4, which serves, among other things, as an applicator surface for the material to be applied. This applicator surface 4 also has, when projected onto a plane, a drop-shaped, egg-shaped, or pear-shaped contour line 5 that extends on all sides beyond the imaginary extension of the stalk 2. Moreover, in the embodiment shown, the applicator surface 4 is continuously convexly curved in the direction along an imaginary line 6 between two extreme points EX1 and EX2.

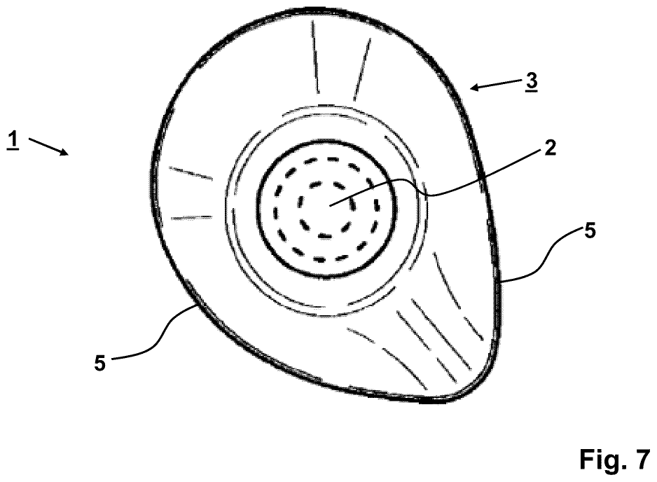

FIG. 7 clearly illustrates what is meant by “projected onto a plane,” as the typical “egg shape” of the applicator element 3 can be seen here from the bottom view in a horizontal plane.

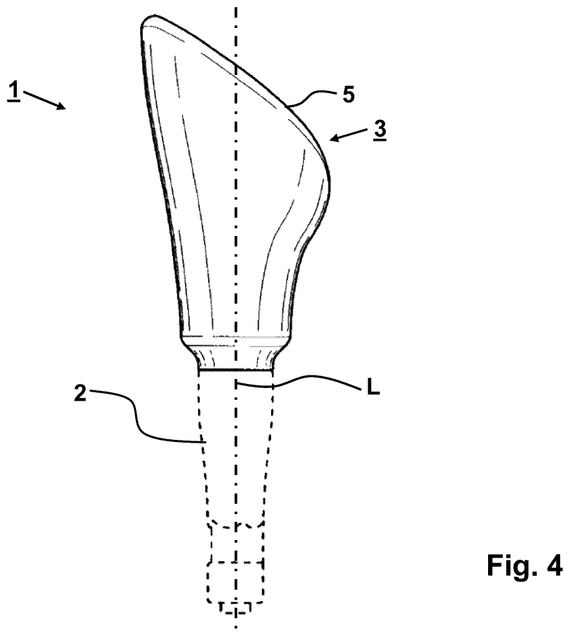

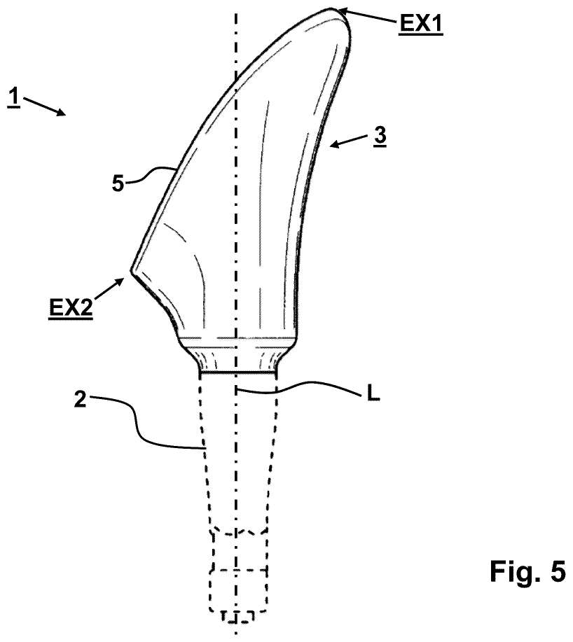

FIGS. 4 and 5 also show the previously mentioned shoe shape, which is the particularly preferred design of the applicator element 3.

The “cross stripes” shown in the figures on the applicator surface 4 are primarily intended as directional indicators—to better illustrate the curvature and bending of the applicator surface 4—and are not necessarily indentations in the applicator surface 4, although such indentations can also be desirable in some cases.

### Second Embodiment

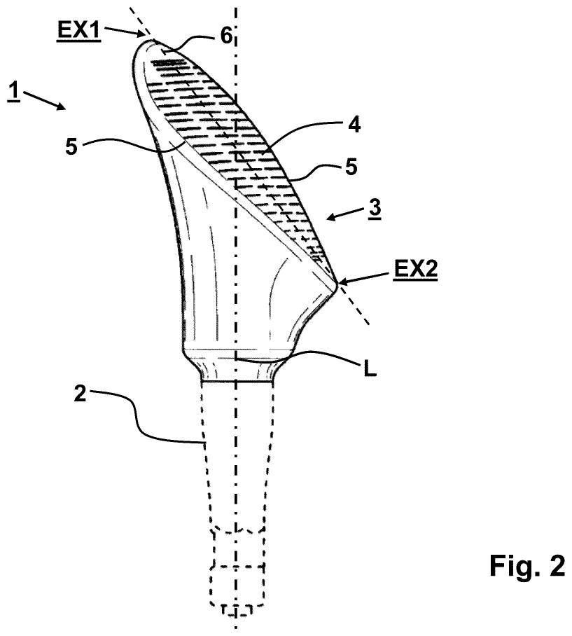

FIG. 2 shows a second preferred exemplary embodiment of the applicator 1 according to the disclosure. The applicator 1 comprises a stalk 2, which is indicated only by dashed lines. This stalk 2 can serve as an additional part for attaching a handle or can itself be the handle that the user holds in the hand or between the fingers to use the applicator 1 as intended. The central longitudinal axis L of the applicator 1 is the central longitudinal axis of the stalk 2, extended imaginatively.

Projecting from the stalk 2, the applicator 1 further comprises an applicator element 3, which carries the applicator surface 4.

On its front side facing away from the stalk 2, the applicator 1 has an applicator surface 4, which is used, among other things, as an applicator surface for the material to be applied. This applicator surface 4 also has, when projected onto a plane, a drop-shaped, egg-shaped, or pear-shaped contour line 5 that extends on all sides beyond the imaginary extension of the stalk 2. Moreover, in the embodiment shown, the applicator surface 4 is continuously flat in the direction along an imaginary line 6 between two extreme points EX1 and EX2.

FIG. 7 clearly illustrates what is meant by “projected onto a plane,” as the typical “egg shape” of the applicator element 3 can be seen here from the bottom view in a horizontal plane.

FIGS. 4 and 5 also show the previously mentioned shoe shape, which is the particularly preferred design of the applicator element 3.

The “cross stripes” shown in the figures on the applicator surface 4 are primarily intended as directional indicators—to better illustrate the curvature and bending of the applicator surface 4—and are not necessarily indentations in the applicator surface 4, although such indentations can also be desirable in some cases.

### Third Embodiment

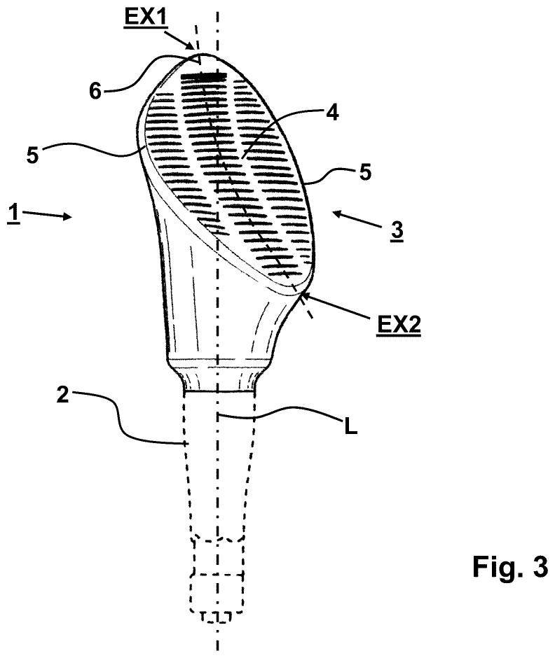

FIG. 3 shows a third preferred embodiment of the applicator 1 according to the disclosure. The applicator 1 comprises a stalk 2, which here is indicated only by dashed lines. This stalk 2 can serve as an additional part for attaching a handle or can itself be the handle that the user holds in the hand or between the fingers to use the applicator 1 as intended. The central longitudinal axis L of the applicator 1 is the central longitudinal axis of the stalk 2, extended imaginatively.

Projecting from the stalk 2, the applicator 1 further comprises an applicator element 3, which carries the applicator surface 4.

On its front side facing away from the stalk 2, the applicator 1 namely has an applicator surface 4 that serves, among other things, as the applicator surface for removing the material to be applied. This applicator surface 4 also has, when projected onto a plane, a drop-shaped, egg-shaped, or pear-shaped contour line 5 that extends on all sides beyond the imaginary extension of the stalk 2. Moreover, in the embodiment shown, the applicator surface 4 is continuously concavely curved in the direction along an imaginary line 6 between two extreme points EX1 and EX2.

FIG. 7 clearly illustrates what is meant by “projected onto a plane,” as the typical “egg shape” of the applicator element 3 can be seen here from the bottom view in a horizontal plane.

FIGS. 4 and 5 also show the previously mentioned shoe shape, which is the particularly preferred design of the applicator element 3.

The “cross stripes” shown in the figures on the applicator surface 4 are primarily intended as directional indicators—to better illustrate the curvature and bending of the applicator surface 4—and are not necessarily indentations in the applicator surface 4, although such indentations can also be desirable in some cases.

### Fourth Embodiment

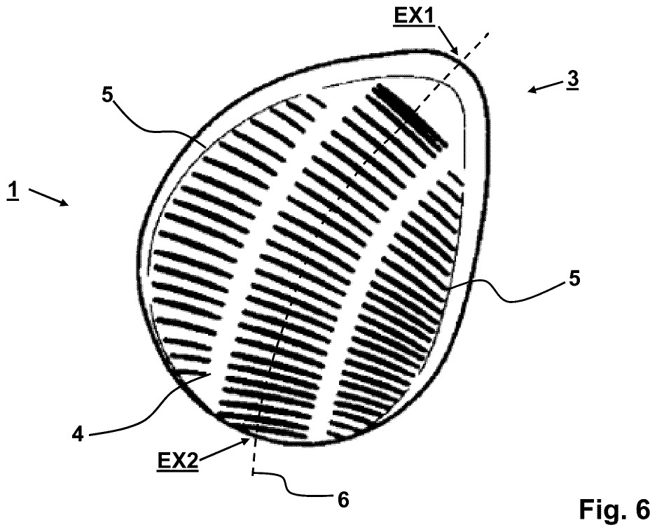

FIG. 6 shows another possible design of the applicator element 3. Here, the two extreme points EX1 and EX2 are not directly opposite each other, meaning the applicator 1 is not mirror-symmetrical. The extreme points are instead arranged offset from one another, so that when viewed from above, an imaginary line 6 connecting the extreme points is not straight. The applicator surface 4 in this embodiment essentially forms a twisted plane.

This configuration can additionally be applied to all three previously shown embodiments.

An applicator 1 can generally be designed so that it consists of at least two different materials.

However, it is also possible for an applicator 1 to be made entirely of the same material.

An applicator 1 can also include additional bristles. These bristles are preferably arranged on the outer side of the applicator surface 4. The bristles are preferably oriented perpendicularly to their base.

An applicator according to the disclosure can also be manufactured from post-consumer recycled material. Thus, even the various materials of the applicator 1 can be post-consumer recycled materials.

The applicator 1 is preferably suitable for the application of medical, cosmetic, pharmaceutical, or dental products.

It should again be noted that the applicator surface 4 is not the only part of the applicator element 3 intended for application purposes. In many cases, it is preferable that the application process—and especially precise “fine-tuning”—can be carried out using the entire applicator element 3. In particular, the circumferential area of the applicator element 3 extending from the stalk toward the applicator surface 4 can be used for applying material. The area adjoining the first extreme point EX1 is especially well-suited for controlled application and distribution of material.

The transition from the applicator surface 4 to the aforementioned circumferential area is preferably a transition with a rounding, but in certain cases can also be angular. This allows for various application characteristics of the applicator element 3 to be achieved.

What is claimed is:

1. An applicator for applying a cosmetic or pharmaceutical product, comprising:
a stalk; and
an applicator element attached to the stalk, the applicator element having, on a front side facing away from the stalk, an applicator surface which, when projected onto a plane, has a cam-shaped, drop-shaped, egg-shaped, or pear-shaped contour line extending on all sides beyond an imaginary extension of the stalk and is continuously convexly curved, concavely curved, or flat in a direction along an imaginary line between first and second extreme points.

2. The applicator according to claim 1, wherein the contour line of the applicator surface has smallest curvature at the first extreme point and a second smallest curvature at the second extreme point.

3. The applicator according to claim 1, wherein a curvature of the contour line at the second extreme point is at least three times greater than a curvature of the contour line at the first extreme point.

4. The applicator according to claim 1, wherein the first and second extreme points are positioned diametrically opposite each other with respect to an extended imaginary central longitudinal axis of the stalk.

5. The applicator according to claim 1, wherein the applicator surface is less curved, not curved, or at least substantially not curved in the a perpendicular to the imaginary line between the first and second extreme points.

6. The applicator according to claim 1, wherein the applicator is formed entirely or partially of a TPE, a TPO, or a mixture containing TPE or TPO.

7. The applicator according to claim 1, wherein the applicator surface or an entirety of the applicator element is partially or completely coated with a different plastic than that forming a main body of the applicator element.

8. The applicator according to claim 1, wherein a Shore hardness D of the applicator surface or an entirety of the applicator element is less than or equal to 56.

9. The applicator according to claim 1, wherein the applicator surface or an entirety of the applicator element carries a flocking or micro-bristles or another type of surface modification.

10. The applicator according to claim 1, wherein the applicator surface and an edge thereof or an entirety of the applicator element are completely free of bristles.

11. The applicator according to claim 1, wherein a tip of the applicator surface in an area of the first extreme point is shaped and made of a material such that, under forces occurring during normal use, the tip is capable of moving by at least 2 mm relative to a remainder of the applicator element.

12. The applicator according to claim 1, wherein the applicator element is designed in a form of a shoe.

13. The applicator according to claim 12, wherein the applicator element widens uniformly upward from the stalk, then widens more on a first side than on a second side diametrically opposite with respect to the extended longitudinal axis.

14. The applicator according to claim 13, wherein the first side ends lower in a direction of the stalk at the first extreme point, than the second side.

15. The applicator according to claim 14, wherein, the second side ends at the second extreme point, and the applicator surface spans between the first and second sides and between the first and second extreme points.

16. The applicator according to claim 1, wherein a tip of the applicator surface in an area of the first extreme point is shaped and made of a material such that, under forces occurring during normal use, the tip is capable of pivoting by at least 2 mm relative to a remainder of the applicator element.
